(19) United States
(12) Patent Application Publication
(10) Pub. No.: US 2021/0223205 A1
(43) Pub. Date: Jul. 22, 2021

(54) HIGH-SENSITIVITY MOLECULAR DETECTING DEVICE EMPLOYING METAL ION ENCAPSULATED FULLERENE

(71) Applicant: IDEA INTERNATIONAL INC., Sendai-shi Miyagi (JP)

(72) Inventors: Kazuhiko KAWACHI, Sendai-shi Miyagi (JP); Shoichi ONO, Sendai-shi Miyagi (JP); Tomoo KAMIGAKI, Sendai-shi Miyagi (JP); Yasuhiko KASAMA, Sendai-shi Miyagi (JP)

(21) Appl. No.: 17/044,338

(22) PCT Filed: Apr. 1, 2019
(86) PCT No.: PCT/JP2019/014552

§ 371 Date: Dec. 7, 2020

(30) Foreign Application Priority Data
Apr. 2, 2018 (JP) 2018-071188

(51) Int. Cl. G01N 27/66; G01N 33/497

(52) U.S. Cl. CPC G01N 27/66; G01N 33/497

(57) ABSTRACT

The objective of the present invention is to provide a high-sensitivity molecular detecting device using metal ion encapsulated fullerene capable of detection even at ppt level concentrations.

This high-sensitivity molecular detecting device using metal ion encapsulated fullerene includes
a container having an introduction port for introducing detected molecule into the main body of the container,
complex of dye and metal ion encapsulated fullerene contained inside the container,
a pair of electrodes,
a light radiating means for irradiating the inside of the container with light, and
an ammeter for measuring a current flowing between the electrodes; and
an electron orbit energy level is set such that there is no electron movement in a ground state without light irradiation, and electron separated from the detected molecule moves into vacancy generated by means of an excitation in an excited state resulting from light irradiation.

### TECHNICAL FIELD

The present invention relates a high-sensitivity molecular detecting device using metal ion encapsulated fullerene

### TECHNICAL FIELD

Early detection of cancer is desired. For example, it is known that the exhaled breath of a cancer patient contains a substance, which is not contained in the exhaled breath of a person who is not a cancer patient.

For example, patients with esophageal cancer are known to be rich in four types of molecules: 2-butanone, acetic acid, acetone, and acetonitrile. For other cancers, other molecules are contained. In addition, it is expected that many molecules will be found in clinical practice in the future.

The molecules shown in FIG. 3 are gas substrates, which are abundantly contained in exhaled breath of a lung cancer patient.

And, it is attempted to detect cancer at an early stage by utilizing the detection of such a substance. Currently, for example, the following techniques are being attempted as detection techniques. (1) A technology, in which gas is adsorbed on a functional film applied to a Si diaphragm formed by MEM, and then the mechanical strain of the functional film generated by the adsorption is detected as an electric current (cantilever method). (2) A technology for detecting changes in resistance due to adsorption of gas molecules (semiconductor sensor method). (3) A technology, which detects fluorescence by passing odors through molecules to the olfactory receptors of genetically engineered mice (biochip method). (4) A technology, which utilizes the fact that when a dilute gas is passed through a fluctuating electric field, only molecules with a specific mass cross-sectional area ratio reach the detector (symmetric field ion transfer analysis method).

On the other hand, as a technique using the encapsulated fullerene, the present inventors provide a photoelectric conversion device using the encapsulated fullerene described in Patent Document 1. The device is a photoelectric conversion device using an encapsulated fullerene containing supramolecular complex composed of alkaline ion-encapsulating fullerene and anionic dye.

### PRIOR ART DOCUMENTS

### Patent Documents

PATENT DOCUMENT 1 JP 2015-69731 A

### DISCLOSURE OF THE INVENTION

### Problems to be Solved by the Invention

According to the detection techniques (1) to (4) described above, the detection concentration is at the ppb level at best. In addition, the technology of (4) requires a large-scale device.

In the early stage of cancer, a more sensitive detection technique is desired considering that the concentration of substances contained in exhaled breath is also very small.

An object of the present invention is to provide a high-sensitivity molecular detecting device using a metal ion encapsulated fullerene in order to be able to detect substances even at a concentration of ppt level.

### Solutions for Solve the Problems

The invention according to claim 1 is a high-sensitivity molecular detecting device using metal ion encapsulated fullerene;

including

a container with an introduction port for introducing detected molecule into the main body of the container,

complex of dye and metal ion encapsulated fullerene contained inside the container.

a pair of electrodes,

a light radiating means for irradiating the inside of the container with light, and

an ammeter for measuring a current flowing between the electrodes;

wherein an electron orbit energy level is set such that there is no electron movement in a ground state without light irradiation, and electron separated from the detected molecule moves into vacancy generated by means of an excitation in an excited state resulting from light irradiation.

The invention according to claim 2 is the high-sensitivity molecular detecting device using metal ion encapsulated fullerene according to claim 1, wherein the metal is alkali metal.

The invention according to claim 3 is the high-sensitivity molecular detecting device using metal ion encapsulated fullerene according to claim 2, wherein the alkali is Li.

The invention according to claim 4 is the high-sensitivity molecular detecting device using metal ion encapsulated fullerene according to any on of claims 1 to 3, the fullerene is C₆₀.

The invention according to claim 5 is the high-sensitivity molecular detecting device using metal ion encapsulated fullerene according to claim 3, wherein the ion encapsulated fullerene is Li⁺@C₆₀.

The invention according to claim 6 is the high-sensitivity molecular detecting device using metal ion encapsulated fullerene according to any one of claims 1 to 5, wherein the dye is a macromolecular polymer such as polythiophene poly-3-hexylthiophene (P3HT) and so on, poly p-phenylene, poly p-phenylene vinylene, polyaniline, polypyrrole, PEDOT, P3OT, POPT, MDMO-PPV, or MEH-PPV; or their derivatives.

### Effects of the Invention

Despite its extremely simple structure, for example, molecules specifically contained in the exhaled breath of a cancer patient can be detected even at a concentration of ppt level.

The detection target is not limited to molecules specifically contained in the exhaled breath of cancer patients, but can be any gas, solid, or liquid.

### BRIEF DESCRIPTION OF THE DRAWINGS

FIG. 1 shows a conceptual perspective diagram of a high-sensitivity molecular detecting device using metal ion encapsulated fullerene according to the embodiment of the present invention.

FIG. 2 shows a conceptual diagram for explaining the movement of electrons.

FIG. 3 shows a diagram showing gas substrates abundantly contained in exhaled breath of a lung cancer patient.

### DESCRIPTION OF REFERENCE NUMERALS AND SIGNS

1 main body

2 dye

3 metal ion encapsulated fullerene

5, 6 electrode

7 exhaled breath containing molecule to be detected

8 introduction port

### MODE FOR CARRYING OUT THE INVENTION

Complex of electron donor and metal ion encapsulated fullerene constitute donor acceptors, and by light irradiation they generate charge-separated state and possess oxidizing power. In Patent Document 1, a photoelectric conversion device is configured by using this oxidizing power.

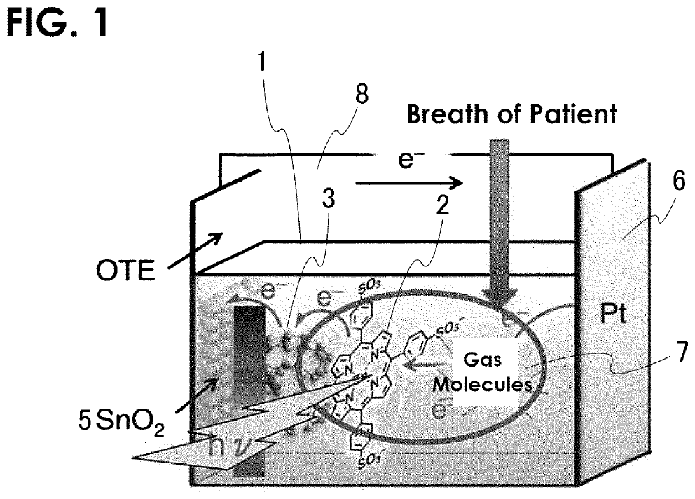

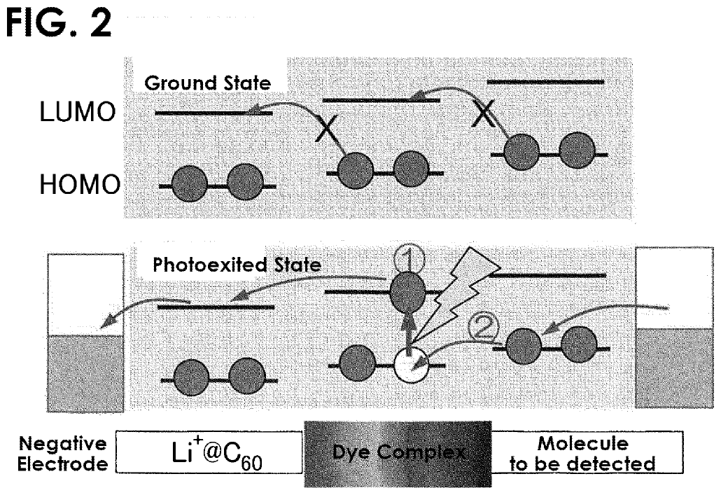

The present inventors examined an use of such characteristics in other devices, and found that it is possible to detect molecules by designing an energy level. That is, as shown in FIG. 1, a basic configuration is a configuration, in which supramolecular complex of electron donor 2 and metal ion encapsulated fullerene 3 is placed inside a main body 1 and a pair of electrodes 5 and 6 are placed sandwiching the supramolecular complex (This configuration is almost the same as the photoelectric conversion device.). However, It was found that when an energy level of the complex of the electron donor and the metal ion encapsulated fullerene is, for example, set to the state shown in FIG. 2 according to an energy level of the detected molecule and light of a predetermined wavelength is irradiated, electrons separated from molecules can be captured almost one-to-one.

In the example shown in FIG. 1, the electrodes are transparent electrodes and consist of SnO₂. In this example, one electrode uses Pt.

In FIG. 2, HOMO (Highest Occupied Molecular Orbital) and LUMO (Lowest Unoccupied Molecular Orbital) are set as follows.

(d)<(HOMO of dye)<(HOMO of molecule to be detected)

(LUMO of metal ion encapsulated fullerene)<(LUMO of dye)<(LUMO of molecule to be detected)

(LUMO of metal ion encapsulated fullerene)>(HOMO of dye)

(LUMO of dye)>(HOMO of molecule to be detected)

In such a setting, in the ground state (upper part of FIG. 2), electron transfer does not occur even if the molecule to be detected is introduced.

On the other hand, in the excited state, that is, in the state of being irradiated with light as shown in the lower part of FIG. 2, the electron of HOMO is excited and move to the level of LUMO in the dye, and vacancy is generated in HOMO. That is, a charge-separated state is generated and possesses acid HOMO forming ability.

In the photoexcited state, if the molecule to be detected is ionized, the separated electron moves to the vacancy of the dye. On the other hand, the electron excited in the dye moves to the LUMO of the metal ion encapsulated fullerene, and an electric current flows.

It is assumed that 500 cc of exhaled breath contains only 1 ppt of the molecule to be detected. The number of molecules contained in the exhaled breath of 500 cc is

5 L/25.36 L×6.02×1023÷1.19×1022

If it is assumed that only 1 ppt contains the molecule to be detected, the number of molecules is

1.19×10²²×10⁻¹²÷1.19×10¹⁰

The amount of charge released by one-electron oxidation is

6×10⁻¹⁹ C×1.19×10¹⁰÷1.9×10⁻⁹ C

If this amount of electric charge is passed in 1 second, 1.9 nA=10 pA.

Whereas other detection techniques indirectly convert (for example, from distortion) into an electrical signal, the present invention steals electrons from the molecule to be detected and directly converts them into electrical signals without changing to mechanical quantities. Due to the change, an extremely sensitive detector is achieved.

Examples of the dye include π-electron compounds, for example, porphyrins, metal chelate compounds, polyaniline compounds, aromatic polycyclic compounds, and compounds having a polyacene-based skeleton structure.

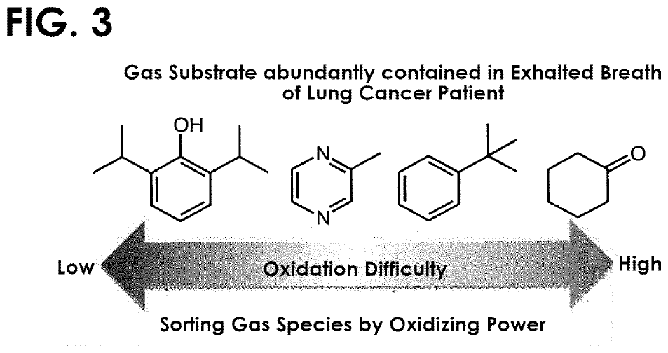

These energy levels can be easily determined by a molecular orbital method. The energy level of the molecule shown in FIG. 3 can also be easily known.

In the molecular orbital method, the atomic orbitals of each atom are combined to create a one-electron molecular orbital, and it is optimized to obtain the one-electron molecular orbital with the highest approximation. And, two molecules (with their spins reversed) are stored in order from the one-electron molecular orbital with the lowest energy, and all the electrons of that molecule are stored. Further, the spatial distribution of the establishment of the existence of electrons is calculated, and how the electrons are spread around the molecule is clarified. In this way, the electronic state of the molecule can be known. However, the excitation energy of the molecule and the HOMO-LUMO gap do not always match. The HOMO and the LUMO are qualitatively determined by the molecular orbital method, and more exactly, it can be confirmed whether the energy level relationship shown in FIG. 2 is achieved by constructing the device shown in FIG. 1 using known molecules to be detected and checking whether a current flows. On the contrary, the HOMO and LUMO states of the dye can be known.

The complex of the dye and the metal ion encapsulated fullerene may be retained in the container main body 1 together with the appropriate solvent. Further, it is preferable that the metal ion encapsulated fullerene is on the electrode 5 side.

### INDUSTRIAL APPLICABILITY

Although the present invention is optimally applied to a medical field, it can be applied not only in the field but also in any field where substance detection is required.

1. A high-sensitivity molecular detecting device using metal ion encapsulated fullerene;
Including
a container with an introduction port for introducing detected molecule into the main body of the container,
complex of dye and metal ion encapsulated fullerene contained inside the container.
a pair of electrodes,
a light radiating means for irradiating the inside of the container with light, and
an ammeter for measuring a current flowing between the electrodes;
wherein an electron orbit energy level is set such that there is no electron movement in a ground state without light irradiation, and electron separated from the detected molecule moves into vacancy generated by means of an excitation in an excited state resulting from light irradiation.

2. The high-sensitivity molecular detecting device using metal ion encapsulated fullerene according to claim 1, wherein the metal is alkali metal.

3. The high-sensitivity molecular detecting device using metal ion encapsulated fullerene according to claim 2, wherein the alkali metal is Li.

4. The high-sensitivity molecular detecting device using metal ion encapsulated fullerene according to claim 1, the fullerene is C₆₀.

5. The high-sensitivity molecular detecting device using metal ion encapsulated fullerene according to claim 3, wherein the ion encapsulated fullerene is Li⁺@C₆₀.

6. The high-sensitivity molecular detecting device using metal ion encapsulated fullerene according to claim 1, wherein the dye is a macromolecular polymer such as polythiophene poly-3-hexylthiophene (P3HT) and so on, poly p-phenylene, poly p-phenylene vinylene, polyaniline, polypyrrole, PEDOT, P3OT, POPT, MDMO-PPV, or MEH-PPV; or their derivatives.
